(19) United States
(12) Patent Application Publication
(10) Pub. No.: US 2003/0153787 A1
(43) Pub. Date: Aug. 14, 2003

(54) Surfactants

(75) Inventors: Neil Michael Carpenter, Cleveland (GB); Steven John Anderson, Cleveland (GB); Richard Robert Tenore, Northeast, MD (US); Peter Glynn Hibbert, Newark, DE (US)

Correspondence Address: PILLSBURY WINTHROP, LLP, P.O. BOX 10500, MCLEAN, VA, 22102, US

(73) Assignee: Imperial Chemical Industries PLC, London (GB)

(21) Appl. No.: 10/315,210

(22) Filed: Dec. 10, 2002

Related U.S. Application Data
(63) Continuation of application No. 09/383,130, filed on Aug. 25, 1999, now abandoned.
Application No. 09/383,130 is a continuation of application No. PCT/GB98/00562, filed on Feb. 24, 1998.

(30) Foreign Application Priority Data
Feb. 27, 1997 (GB) 9704126.3

(51) Int. Cl. C07C 239/00

(52) U.S. Cl. 562/592; 562/590; 526/932; 564/123; 564/133; 564/138; 564/164

(57) ABSTRACT

Compounds of the formula R².[(AO)ₙ.R³]ₘ, where R² is a residue of a group having at least m active hydrogen atoms derived from hydroxyl and/or amino and/or amido groups, AO is alkyleneoxy, n is 2 to 200; R³ includes residue(s) of alkenyl succinic acids and optionally other acids, and m is 2 to 10, but when m is 2 there are other restrictions in the definitions, are disclosed as useful thickeners and/or dispersants in aqueous systems. The use of such materials as thickeners is also disclosed.

This invention relates to derivatives of substituted succinic acids and to the use of such derivatives particularly as thickeners especially in personal care compositions, especially such compositions including large proportions of other surfactants, especially detergent surfactants as for example used in shampoos, particularly baby shampoos.

Some derivatives of substituted succinic acids are described in EP 0107199 B as useful surfactants under acid conditions and published PCT Application No WO 94/00508 A describes surfactants based on alk(en)yl substituted succinic acid alkylene oxide esters and amides. Subsequently published PCT Applications describe the use of groups of this class of surfactant in various end use applications: in WO 95/06070 A as emulsifiers in oil in water emulsion polymerisation, in WO 95/06096 A as detergents in so-called hard surface cleaning, in WO 95122896 A as emulsifiers in agrochemical formulations and in WO 95/22897 A as adjuvants in agrochemical formulations.

We have now found that esters of alkyl or alkenyl succinic acids with polyalkylene oxide derivatives of polyhydroxyl compounds in which there are two or more and especially three or more ester groups including alkenyl succinic acid ester groups, in particular where there are three or more alkyl or alkenyl succinic acid ester groups, can have very useful thickening and or dispersant properties.

This invention accordingly provides compounds of the formula (1):

R².[(AO)ₙ.R³]ₘ (1)

where:

R² is the residue of a group having at least m active hydrogen atoms derived from hydroxyl and/or amino and/or amido groups;

AO is an alkylene oxide residue, which may vary along the chain;

each n is from 2 to 200;

m is from 2 to 10; and

each R³ is H, hydrocarbyl, particularly a C₁ to C₂₂ alkyl or alkenyl, a long chain alk(en)yl succinic acyl group of the formula OC.(HR)C.C(HR¹).COY where:

one of R and R¹ in the succinic moiety is C₈ to C₂₂ alkenyl or alkyl and the other is hydrogen, and

Y is a group OM where M is hydrogen, metal, amonium, amine, especially alkylamine (including alkanolamine), onium, hydrocarbyl, desirably C₁ to C₂₂ hydrocarbyl, more particularly alkyl, especially C₁ to C₂₂ alkyl; or

Y is NR⁴R⁵ where R⁴ and R⁵ are each independently hydrogen, hydrocarbyl, particularly alkyl, including substituted hydrocarbyl such as substituted alkyl, particularly hydroxyl substituted hydrocarbyl, especially polyhydroxy hydrocarbyl, such as hydroxyl substituted and especially polyhydroxy substituted alkyl;

or a long chain acyl group —OC.R⁶, where R⁶ is a long chain hydrocarbyl group, particularly a C₈ to C₂₂ alkyl or alkenyl group;

or a short chain acyl group —OC.R⁷, where R⁷ is a short chain hydrocarbyl group, particularly a C₁ to C₇ alkyl or alkenyl group;

where at least two, and desirably at least three, of the groups R³ are long chain acyl groups, and at least one, desirably at least two and particularly at least three, of the long chain acyl groups is/are long chain alkenyl or alkyl succinic group(s);

provided that where R¹ is ethylene glycolyl or propylene glycolyl, m is 2, and both groups R² are alk(en)yl succinic groups, the total of the indices n is at least 120.

In particular, the invention provides compounds of the formula (1a):

R².[(AO)ₙ.R³]m (1a)

where:

R² is the residue of a group having at least m active hydrogen atoms derived from hydroxyl and/or amino and/or amido groups;

AO represents ethylene oxide residues or a mixture of ethylene oxide residues and propylene oxide residues in which the molar proportion of ethylene oxide residues is at least 50% and desirably at least 70%;

each n is from 10 to 200 such that the total of the indices n is at least 120

m is from 2 to 10;

each R³ is H; hydrocarbyl; particularly C₁ to C₂₂ hydrocarbyl, more particularly C₁ to C₂₂ alkyl or alkenyl;

a long chain alk(en)yl succinic acyl group of the formula:

—OC.(HR)C.C(HR¹).COY

where:

one of R and R¹ in the succinic moiety is C₈ to C₂₂ alkenyl or alkyl and the other is hydrogen, and

Y is a group OM where M is hydrogen, metal, amonium, amine especially alkylamine (including alkanolamines), or Y is NR⁴R⁵ where R⁴ and R⁵ are each independently hydrogen, a hydrocarbyl, particularly alkyl, group, including substituted hydrocarbyl such as substituted alkyl, particularly hydroxyl substituted hydrocarbyl, especially polyhydroxy hydrocarbyl, such as hydroxyl substituted and especially polyhydroxy substituted alkyl, groups;

a long chain acyl group —OC.R⁶, where R⁶ is a long chain hydrocarbyl group, particularly a C₈ to C₂₂ alkyl or alkenyl group; or

a short chain acyl group —OC.R⁷, where R⁷ is a short chain hydrocarbyl group, particularly a C₁ to C₇ alkyl or alkenyl group;

where at least two, and desirably at least three, of the groups R³ are long chain acyl groups, and at least one, desirably at least two and particularly at least three, of the long chain acyl groups is/are long chain alkenyl or alkyl succinic group(s).

In addition to the compounds of the invention themselves, we have found that related compounds (including some compounds falling within the definitions in our earlier WO 94/00508 A) can also successfully be used as thickeners. Accordingly, the invention includes, the use as thickeners, of compounds of the formula (II):

R¹².[(AO²)ₙ₂.R¹³]ₘ₂ (II)

where:

R¹² is the residue of an optionally substituted hydrocarbyl group having at least m active hydrogen atoms derived from hydroxyl and/or amino and/or amido groups;

AO² is an alkylene oxide residue, which may vary along the chain;

each n2 is from 10 to 200, such that the total of the indices n2 is at least 50;

m2 is from 2 to 10; and

each R¹³ is H, hydrocarbyl, particularly a C₁ to C₂₂ alkyl or alkenyl,

a long chain alk(en)yl succinic acyl group of the formula:

—OC.(HR¹⁰)C.C(HR¹¹).COY²

where:

one of R¹⁰ and R¹¹ in the succinic acid moiety is C₈ to C₂₂ alkenyl or alkyl and the other is hydrogen, and

Y² is a group OM² where M² is hydrogen, metal, amonium, amine especially alkylamine (including alkanolamines) alkyl, especially C₁ to C₂₂ alkyl; or

Y² is NR¹⁴R¹⁵ where R¹⁵ and R¹⁵ are each independently hydrogen, a hydrocarbyl, particularly alkyl group, including substituted hydrocarbyl such as substituted alkyl, particularly hydroxyl substituted hydrocarbyl, especially polyhydroxy hydrocarbyl, such as hydroxyl substituted and especially polyhydroxy substituted alkyl, groups;

or a long chain acyl group —OC.R¹⁶, where R¹⁶ is a long chain hydrocarbyl group, particularly a C₈ to C₂₂ alkyl or alkenyl group;

or a short chain acyl group —OC.R¹⁷, where R¹⁷ is a short chain hydrocarbyl group, particularly a C₈ to C₇ alkyl or alkenyl group;

where at least two of the groups R¹³ are long chain acyl groups, and at least one of the long chain acyl groups is a long chain alkenyl or alkyl succinic group.

The compounds of and used in the invention have shown promise as thickeners in various systems, particularly those involving aqueous phases, mainly but not exclusively aqueous continuous phases. The invention accordingly includes the use of the compounds of the formula (I) as defined above as thickeners, in particular as thickeners in oil-in-water and water-in-oil emulsions, aqueous solutions and dispersions of solids in aqueous systems and as emulsifiers, especially as co-emulsifiers or emulsion stabilisers in combination with other surfactants. In particular, in this aspect the invention specifically includes, the use as thickeners, of compounds of the formula (1a) as defined above. The invention further includes oil-in-water and water-in-oil emulsions, aqueous solutions and dispersions of solids in aqueous systems and which include at least one compound of the formula (I) as defined above as a thickener. In particular, in this aspect the invention specifically includes, oil-in-water and water-in-oil emulsions, aqueous solutions and dispersions of solids in aqueous systems and which include at least one compound of the formula (1a) as defined above in an amount to provide effective thickening of the system.

The compounds of and used in the invention are particularly useful in the thickening of aqueous systems containing other surfactants as in cleaning products especially shampoos and similar products. Conventional shampoos, particularly mild shampoos such as baby shampoos, thickened with conventional thickeners, particularly polyethylene glycol (PEG) distearates e.g. PEG 6000 distearate, tend to show near Newtonian flow behavior, in particular they are not substantially shear thinning. In consequence, the shampoos are made to heave relatively low viscosities, and are thus difficult to handle, so that they will not exhibit the ‘gel-ball’ effect when rubbed between the hands. In this invention, the thickeners show significant shear thinning and this enables shampoo formulations to be made having higher viscosity, so that it is easier to handle, but that do not ‘ball up’ when rubbed in the hands or hair because they are shear thinning.

The compounds of and used in the invention have shown promise as dispersants in various systems, particularly those involving dispersion of solids in aqueous phases. The invention accordingly includes the use of the compounds of the formula (I) as defined above as dispersants, particularly as dispersants for solids in aqueous phases, especially pigment solids in aqueous phases. The invention further includes dispersions of solids in aqueous systems which include at least one compound of the formula (I) as defined above as a dispersant. In particular, in this aspect the invention specifically includes aqueous dispersions of solids which include at least one compound of the formula (Ia) as defined above in an amount to provide effective dispersion of the solid in the aqueous phase.

Among the uses within the invention, the invention specifically includes oil-in-water and water-in-oil emulsions, aqueous solutions and dispersions of solids in aqueous systems and which include at least one compound of the formula (II) (including compounds of the formula (I) or formula (Ia)) as defined above as a thickener.

The compounds of and used in the invention are at least notionally built up from the group R² or R¹² which can be considered as the “core group” of the compounds. This core group is the residue (after removal of m active hydrogen atoms) of a compound containing at least m active hydrogen atoms in hydroxyl and/or amino and/or amido groups. Usually it is the residue of an optionally substituted hydrocarbyl group, particularly a C₃ to C₃₀ hydrocarbyl compound. At its simplest the core group can be an ethylene glycolyl (—O.C₂H₄.O—) or propylene glycolyl (—O.C₃H₆.O—) group, in which case, when the group AO (or AO²) is an ethyleneoxy or propyleneoxy group, the core group can (notionally) be considered as being any of the ethylene or propylene glycolyl groups along the chain. For convenience, the (or a) group near the middle of the chain will be considered as being the core group in this case and in this case (when further both end acyl groups are substituted succinic groups), among the compounds of the invention the total number of ethyleneoxy and propyleneoxy groups is at least 120, although the number can be lower than this in the method and use of the invention. However, the core group will often be a residue where at least 3 active hydrogen atoms have been removed.

Examples of core groups include the residues of the following pounds after removal of at least two active hydrogen atoms:

1 glycerol and the polyglycerols, especially diglycerol and triglycerol;

2 tri- and higher polymethylol alkanes such as trimethylol ethane, trimethylol propane and penterythritol;

3 sugars, particularly non-reducing sugars such as sorbitol and mannitol, etherified derivatives of sugars such as sorbitan (the cyclic dehydro-ethers of sorbitol), partial alkyl ethers of sugars such as methyl glucose and alkyl (poly-)saccharides, and ether oligo-/poly-mers of sugars such as dextrins, esterified derivatives of sugars such as fatty acid esters such as the fatty acid e.g. lauric, palmitic, oleic, stearic and behenic acid, esters of sorbitan and the sorbitols (themselves well known as surfactants and which when alkoxylkated with ethylene oxide form the well known polysorbate group of surfactants in which at least part of the ethoxylation is effectively inserted between the fatty acid residue and the sorbitol residue), (non-reducing sugars being preferred over reducing sugars as they are more stable under typical synthetic conditions and tend to give products which are less susceptible to oxidation and are less highly coloured—colour mainly arising from oxidative degradation);

4 polyhydroxy carboxylic acids especially citric and tartaric acids;

5 amines including di- and poly-functional amines, particularly alkylamines including alkyl diamines such as ethylene diamine (1,2-diaminoethane);

6 amino-alcohols, particularly the ethanolamines, 2-aminoethanol, di-ethanoiamine and triethanolamine;

7 carboxylic acid amides such as urea, malonamide and succinamide; and

8 amido carboxylic acids such as succinamic acid.

The index m is a measure of the functionality of the core group and generally the alkoxylation reactions will replace all active hydrogen atoms in the molecule from which the core group is derived. (Of course, reaction at a particular site may be inhibited by suitable protection.) The terminating hydroxyl groups of the polyalkylene oxide chains in the resulting compounds are then available for reaction with acyl compounds to form ester linkages and other compounds (if desired) (see below). The index m will typically be in the range 2 to 10, more usually from 2 and especially 3 to 6.

The alkylene oxide groups AO and AO² are typically groups of the formula: —(CₘH₂ₘO)— where m is 2, 3 or 4, desirably 2 or 3, i.e. an ethyleneoxy (—C₂H₄O) or propyleneoxy (C₃H₆O—) group, and it may represent different groups down the alkylene oxide chain. Generally, it is desirable that the chain is a homopolymeric ethylene oxide chain. However, the chain may be a homopolymer chain of propylene glycol residues or a block or random copolymer chain containing both ethylene glycol and propylene glycol residues. Usually, where co-polymeric chains of ethylene and propylene oxide units are used the molar proportion of ethylene oxide units used will be at least 50% and more usually at least 70%.

For thickener applications, especially in aqueous systems, the number of alkylene oxide residues in the polyalkylene oxide chains, i.e. the value of the each parameter n. and n2, will generally be from 15 to 150, particularly 20 to 120, especially 50 to 100. In practice, in compounds of and used in this invention the total degree of alkoxylation may be a useful guide to satisfactory thickener properties. Thus, desirable compounds include those where the total of the indices n is from 30 to 300, particularly 50 to 250, especially 80 to 200.

For dispersant applications, especially the dispersion of solids such as pigments in aqueous systems, the number of alkylene oxide residues in the polyalkylene oxide chains, i.e. the value of the each parameter n and n2, will generally be from 10 to 100, particularly 20 to 80, especially 40 to 70. The total degree of alkoxylation (the total of all the indices n and n2) will typically be from 30 to 300, particularly 50 to 250, especially 80 to 200.

In any particular product, the value of the index n or n2 is an average value which includes statistical variation in the chain length between the same substituent in different molecules and between different substituent groups. For use as thickeners in mainly aqueous systems, the compounds of and used in the invention desirably have a molecular weight of at least 4000 D and typically not more than about 8000 D. For use as dispersants, the molecular weight will typically be from 1000 to 4000 D.

The groups R³ and R¹³ are the “terminating groups” of the polyalkylene oxide chains. For practical thickener use at least two of the terminating groups will be acyl groups and desirably at least two of the terminating groups are alk(en)yl succinic groups as defined above in formuale (I), (Ia) or (II). Where R³ and R¹³ are alk(en)yl succinic groups as defined above the one of R and R¹ which is a C₈ to C₂₂ alkyl or alkenyl group is particularly a C₁₆ or longer group. For dispersant use, generally at least two of the terminating groups are alk(en)yl succinic groups as defined above in formuale (I), (Ia) or (II). Where R³ and R¹³ are alk(en)yl succinic groups as defined above the one of R and R¹ which is a C₁₂ to C₁₈ alkyl or alkenyl group is particularly a C₁₂ to C₁₈ group.

The number of terminating groups may exceed the number of acyl groups and in this case, the remaining terminating groups can be hydrogen atoms or hydrocarbyl, particularly alkyl, groups. Further, within alk(en)yl succinic terminating groups as defined above in formuale (I), (Ia) or (II) the groups Y and Y² may be hydrocarbyl, particularly alkyl, groups. Suitable such hydrocarbyl groups include lower alkyl groups, e.g. C₁ to C₆ alkyl groups such as methyl or ethyl groups, acting as chain end caps for one or more of the polyalkylene oxide chains mainly to alter the degree of hydrophilicity of the compounds, and longer chain alkyl or alkenyl groups e.g. C₈ to C₂₂ and particularly C₁₆ or longer, alkyl or alkenyl groups such as lauryl, oleyl and stearyl groups or mixed alk(en)yl groups derived from natural fats or oils or from distillation cuts in petrochemical synthesis, acting as secondary hydrophobe(s) in the molecule.

It can be desirable to avoid the presence of free (unreacted) anhydride in compounds of and used in the invention, especially where the intended use of the compounds is in personal care applications. In view of the ease of carrying out the esterification reaction with the anhydrides, residual free anhydride is likely only where the nominal product has no remaining available reactive hydrogens. A convenient way to do this, especially on the small scale, is to use slightly less than the stoichiometric proportion of anhydride corresponding to complete reaction. This is particularly useful where the number of succinic ester functions in the target molecule is 3 or more. (This can be seen in some of the Examples below.) On the laboratory scale, the ‘shortfall’ of anhydride will typically be no less than about 5% (molar) and typically about 1 to 3% (molar). In industrial scale production, it is usually easier to drive the reaction more nearly to stoichiometric balance and the ‘shortfall’ may not be required or will typically be less than about 1% (molar).

Where for the groups R³ the number of acyl residues in the molecule is significantly less than m, the distribution of such groups may depend on the nature of the core group and on the extent and effect of the alkoxylation of the core group. Thus, where the core group is derived from pentaerythritol, alkloxylation of the core residue will typically be evenly distributed over the four available sites from which an active hydrogen can be removed and on esterification of the terminal hydroxyl functions the distribution of acyl groups will be close to the expected random distribution. However, where the core group is derived from compounds, such as sorbitol or sorbitan, where the active hydrogen atoms are not equivalent, alkoxylation will typically give unequal chain lengths for the polyalkyleneoxy chains. This may result in some chains being so short that the other (longer) chains exert significant steric effects making esterification at the “short chain” terminal hydroxyl groups relatively difficult. Esterification then will generally preferentially take place at the “long chain” terminal hydroxyl groups. The end result will be a statistical distribution that is not at first sight “random.”

The groups Y and Y² may also be amido groups NR⁴R⁵ or NR¹⁴R¹⁵ in which the substituent groups can be hydrogen, a hydrocarbyl, particularly alkyl group, including substituted hydrocarbyl such as substituted alkyl, particularly hydroxyl substituted hydrocarbyl, especially polyhydroxy hydrocarbyl, such as hydroxyl substituted and especially polyhydroxy substituted alkyl, groups. When one or both of these groups is(are) alkyl it(they) can be lower alkyl groups, e.g. C₁ to C₆ alkyl groups such as methyl or ethyl groups, or longer chain alkyl e.g. C₈ to C₂₂ groups such as lauryl, oleyl and stearyl groups or mixed alkyl groups derived from natural fats or oils or from distillation cuts in petrochemical synthesis, acting as secondary hydrophobe(s) in the molecule.

Where one or more of these substuents is a polyhydroxy substituted hydrocarbyl it is particularly a polyhydroxy alkyl group desirably having a linear carbon chain of from 4 to 7 carbon atoms and at least three hydroxyl groups directly bonded to chain carbon atoms. The group may include substituents, in particular, alkoxy groups e.g. by etherification of further hydroxyl groups or polyalkylene oxide chains, but the group desirably includes at least three free hydroxyl groups including such hydroxyl groups on substituents of the basic chain. Particularly the group is an open chain tetratol, pentitol, hexitol or heptitol group or an anhydro derivative of such a group. Especially desirably, the group is the residue of, or a residue derived from, a reducing sugar, particularly a monosaccharide such as glucose or fructose, a disaccharide such as maltose or palitose or a higher oligosaccharide. Where the group is the residue of, or a residue derived from, a monosaccharide, the saccharide derived group or residue will usually be present as an open chain material. When present such as group will form a secondary hydrophile and as such it will usually be desirable that the hydrophilicity of this group is not unduly reduced. The open chain form of such groups is typically the most hydrophilic form and will thus usually be the form desired. Groups including internal cyclic ether functionality can however be used, if desired, and may be obtained inadvertently if the synthetic route exposes the group to relatively high temperatures or other conditions which promote etherification. Where this group is the residue of, or a residue derived from, an oligosaccharide it can be considered as an open chain mono-saccharide derived group or residue with a saccharide or oligosaccharide substituent. Particularly useful such groups are derived from glycoses and are of the formula: —CH₂.(CHOH)₄CH₂OH, e.g. corresponding to residues from glucose, mannose or galactose. In this case the amido group is conveniently called a glycamine group and the corresponding amides can be called glycamides. Most commonly such a group will be derived from glucose and the corresponding amine and amides are called glucamines and glucamides. As with the amido groups described above any unsubstituted hydrocarbyl group is particularly a short or long chain alkyl group.

Among the compounds of the invention, those where the alk(en)yl group R/R¹ is a C₈ to C₁₈ alkenyl or alkyl group are particularly useful. Generally in use a thickeners in oil-in-water dispersions or emulsions, compounds where R/R¹ is a C₁₂ and especially C₁₄ to C₁₈ alkenyl or alkyl group are especially desirable. Similarly, compounds where the group R or R¹ is an alkenyl group are more desirable than those where the group is alkyl. Compounds where the group R or R¹ is an alkenyl group, particularly a C₈ to C₂₂ alkenyl group and especially a C₁₄ to C₂₀ alkenyl group, form a specific aspect of the invention.

The compounds of the invention can be made by reacting an alkoxylated polyhydric alcohol of the formula: R².[(AO)ₙ.H]ₘ where R², AO, n and m are as defined above, with an alk(en)yl succinic anhydride, and, optionally, a reactive derivative of a fatty acid of the formula H₂OC.R⁶, where R⁶ is as defined above, in molar ratios corresponding to the number of ASA and optional fatty acid residues desired in the product.

Reactions between the alk(en)yl succinic anhydride and the precursor hydroxylic reagent can readily be carried out, with or without catalysts, by bringing the hydroxylic reagent into contact with the alk(en)yl succinic anhydride. Reaction occurs typically, at temperatures below 200° C. and even below 100° C. The reactants will usually be used in at least approximately stoichiometric proportions. Particularly where stoichiometric proportions are used, further purification does not usually appear to be necessary, but can be carried out if desired.

Where an acyl residue is included in the molecule it will usually be introduced by reaction between an appropriate hydroxylic precursor and the corresponding acid or a reactive derivative such as an acyl halide, especially chloride, ester with a short chain alcohol such as methanol or ethanol, or a mixed anhydride, the other acyl reside being of a relatively volatile acid such as acetic acid. The direct reaction between the fatty acid and the hydroxylic precursor can be carried out, with or without catalysts, by heating typically to a temperature of greater than 100° C. Synthesis using reactive derivatives will usually be possible under milder conditions.

The products of the invention are typically a mixture of isomers corresponding to the two senses of the alk(en)yl succinic anhydride ring opening during synthesis. We have noted that the alkenyl or alkyl chain seems to have a minor steric effect on the isomer ratio with the isomer ratio being typically about 60:40, the major isomer arising from nucleophilic attack at the anhydride carbonyl group remote from the alkenyl or alkyl group (probably because of steric hindrance).

The alkenyl succinic anhydride precursors may be produced by reacting maleic anhydride with an olefin having 6 to 22, particularly 8 to 18, carbon atoms, preferably with an excess, for example a 50 to 200% excess, of olefin at a temperature in the range 150 to 400° C. and preferably 180 to 250° C. and removing excess olefin for example by distillation which is suitably carried out under vacuum. No catalyst is necessary, but it is preferred than an antioxidant is present. These anhydrides are well known commercial materials. In alkenyl succinic anhydrides prepared as described above the double bond normally lies in the 2-position in the alkenyl substituent.

To make products where the group R or R¹ is an alkyl group then either the unsaturated products can be hydrogenated or, and preferably, the intermediate alkenyl succinic anhydride can be hydrogenated to give an alkyl succinic anhydride. Typically, hydrogenation of the anhydride is carried out over a hydrogenation catalyst such as Raney nickel or a Pd/C catalyst. Temperatures of from 15 to 100° C. and pressures of up to 200 bar absolute may be used and, if desired a solvent may be present. For example, the hydrogenation reaction on an alkenyl succinic anhydride may be carried out on the neat liquid at 60° C. at 5 bar H₂ pressure using 5% w/w of Pd/C catalyst over a period of for example about 6 hours.

The alkoxylates used in the synthesis can be made by conventional routes. For most simple alkoxylates and polysorbate type compounds these are well known. However, some of the compounds generating the core groups may not be directly alkoxylated as desired. For example, the direct synthesis of a polyethoxylate of pentaerythritol and ethylene oxide is not practical as the pentaerythritol would need to be heated to above 200° C. to melt it and direct ethoxylation at such temperatures is dangerous! This problem can be sidestepped by ethoxylation in a suitable solvent, such as dimethyl glyoxime, or (and particularly if it is desired to avoid solvents) pentaerythritol can first be propoxylated (at about 200° C. under conventional base catalysed conditions) to add about 1 mole of oxypropylene residues per mole of hydroxyl in the pentaerythritol (in practice typically about 3 to 3.5 moles of propylene oxide are added per mole of pentaerythritol). This moderately propoxylated material is typically a liquid at ambient temperature or at superambient temperatures (up to about 150° C. and typically at about 130° C.) and can then be ethoxylated conventionally. Where the overall degree of ethoxylation is above about 10 oxyethylene residues per mole of pentaerythritol the effect of the initial propoxylation does not alter the properties of the product significantly.

Other approaches to this problem include the use of solvents or diluents as carriers for the material to be alkoxylated. Suitable solvents are inert to the alkoxylation conditions and remain liquid at process temperatures and include materials such as dimethyl glyoxime (diglyme). In industrial scale batch production, a portion (‘heel’) of the previous batch may be retained as the solvent/diluent for the raw material in the next batch. Commonly, in a sequence of batch operations, the first batch uses a solvent, and subsequent batches use a heel from the previous batch so that the need to remove the solvent from the product rapidly diminishes. Similarly in continuous processes, particularly where reagents are continuously fed to a reaction vessel holding a relatively large amount of reagents and products, and from which product is continuously withdrawn, the process may be started up using a solvent (often at much less than the rated full capacity of the equipment) and the reaction mix then used as solvent for further raw materials.

Compounds according to the invention have dispersant and/or thickening capabilities. These properties make the compounds of the invention suitable for use as surfactants in dispersing pigments and similar solids in aqueous media, and in thickening dispersions and/or solutions and/or emulsions.

The compounds of this invention can be used as thickeners in a wide variety of systems, particularly aqueous systems. Such application include use as thickeners in emulsion systems of the oil-in-water types. Examples include personal care applications in shampoos, liquid soap and cleanser products and toiletry applications. Accordingly, the invention includes the use of at least one compound of this invention as a thickener in emulsions, especially aqueous oil-in-water and water-in-oil and oil-in-water emulsion systems. The amount of surfactant used in such dispersant applications depends on the materials employed and the concentration of the emulsion required, but will usually be in the range 0.2 to 10%, more usually 0.05 to 5% and particularly 0.1 to 2.5% by weight of the disperse phase of the emulsion. Other end use applications include thickening surfactant formulations. Previously, such systems have been thickened using amine oxide thickeners and replacements have been sought in order to remove any possibility of in situ formation of nitrosamines. The compounds of and used in this invention can be made containing no nitrogen and thus eliminate any risk of nitrosamine formation from this source. Even where the compounds of the invention include nitrogen, it is usually as amide groups which are not readily susceptible to conversion into nitrosamine groups.

The following Examples illustrate the invention including the manufacture and properties of the compounds of the invention and their end uses and the method of the invention. All parts and percentages are by weight unless otherwise specified.

Abbreviations (for compounds supplying core residues):

| | |
|---|---|
| PE | pentaerythritol |
| Tri-gly | triglycerol |
| eth diam | ethylene diamine |

In compounds made in the Synthesis Examples, where the number of ester groups is non integral, the product compounds are described as x(non-integral)-(alkenyl succinic acid)esters.

Materials

| | |
|---|---|
| Tween 20 | polysorbate 20 |
| Miranol C2M | aqueous disodium cocoamphoiacetate, 38% active |
| Steol CS-330 | aqueous sodium laureth sulfate, 29.1% active |
| Dowci1200 | quaternium-15 |
| SLES | sodium lauryl ether sulphate |
| CDMO | N,N-dimethyl-cocoylamine oxide |
| PEG distearate | Kessco brand polyethylene glycol (PEG) 6000 |
| | distearate ester ex Stepan |
| TR92 | TR92 grade titanium dioxide ex Tioxide Ltd |
| Arlatone 1489 | aqueous solution of sodium cocyl isethionate and decyl |
| | glucoside surfactants ex ICI Surfactants |
| Tensiomild HM | disodium laureth sulphosuccinate ex Hickson Manro |
| Tengobetain L7 | cocamidopropyl betaine ex Goldschmidt |
| Germaben II | Preservative ex Sutton Laboratories |
| G 1821 | PEG 6000 distearate ester ex ICI Surfactants |
| Crothix | proprietary thickener ‘pentaerythritol ethoxylate |
| | tetrastearate’ ex Croda |

Hydroxyl values were measured by the general method of ISO 4327 and results are quoted as mg (KOH equivalent).g (product tested)⁻¹.

General viscosity measurements were made using Brookflied viscometers of the type and operated as described in the respective Examples.

### SYNTHESIS EXAMPLES SE1 to SE38

SE1—Di-(dodecenylsuccinic)ester of pentaerythritol 48-ethoxylate Pentaerythritol 48-ethoxylate

A slurry of pentaerythritol (175 g; 1.28 mol) in dimethylglyoxime (diglyme—inert reaction diluent; 328 g) was placed in a 11 autoclave, potassium methoxide (1.24 g) added and the reaction mix vacuum deaerated at 20° C. and vacuum stripped at 80° C. The mixture was heated to 125° C., propylene oxide (246.3 g; 4.25 mol) was added over about 1.5 hours and the mixture allowed to reactat 125° C. overnight. The mixture was then vacuum stripped at 80° C. for 15 minutes to remove unreacted propylene oxide, transferred to a glass distillation flask and the diglyme removed by vacuum distillation at 100° C. The product was 423.3 g (ca 100% of theory) of pentaerythritol condensed with ca 3.3 propylene oxide units (pentaerythritol 3.3 PO).

Pentaerythritol 3.3 PO (480.5 g; 1.46 mol) and potassium hydroxide (5.22 g of a 45% by weight aqueous solution; 2.35 g) were charged to a 21 autoclave, vacuum deaerated and dried by heating at 110° C. for 1 hour at 0.5 bara (250 kPa absolute) while sparging with dry nitrogen gas. The reaction mix was heated to 135° C. and reacted with gaseous ethylene oxide (1025 g; 23.3 mol) fed gradually to the autoclave. At the end of the reaction, 990.7 g of the product were discharged and neutralised with glacial acetic acid. This product had a hydroxyl value of 218.6 mg(KOH).g⁻¹ giving an average molecular weight of ca. 1027 corresponding approximately to pentaerythritol 3.3PO+16EO. Potassium hydroxide (2.34 g) were added to the product remaining in the autoclave (by calculation 514.8 g; 0.5 mol), the reaction mix dried as described above and reacted as described above with further ethylene oxide (701 g; 15.9 mol). A portion (913.6 g) of this product was discharged and neutralised with glacial acetic acid. This product had a hydroxyl value of 93.1 mg(KOH).g⁻¹ giving an average molecular weight of ca. 2410 corresponding approximately to pentaerythritol 3.3PO+48EO. The product remaining in the autoclave could be reacted on in a similar fashion to make other pentaerythritol alkoxylation products such as pentaerythritol 3.3PO+89EO, pentaerythritol 3.3PO+135EO and pentaerythritol 33PO+158EP.

Dodecenylsuccinic anhydride (18.1 g; 68 mmol) was added to pentaerythritol 48-ethoxylate (81.9 g; 34 mmol), made as described above, in a 250 ml three necked flask equipped with a motor driven paddle stirrer, nitrogen line (providing an inert atmosphere) and dropping funnel, the reaction mixture was heated to and held at 100° C. for 6 hours to form the di-(dodecenylsuccinic acid)ester of pentaerythritol 48-ethoxylate in a yield of 100 g, 100% theory. The reaction was monitored using FT-IR and GLC. The C¹³ and H¹ NMR spectra of the ester product (without further purification) confirmed the structure, indicating the absence of anhydride functionality and that the product was the substantially pure title ester.

SE1a—Di-(dodecenylsuccinic)ester of pentaeyrythritol 48-ethoxylate

In a further simplified method the ethoxylated pentaerythritol and dodecenylsuccinic anhydride in a molar ratio of 1:2 were placed in a sealed jar which was heated in an oven at about 100° C. for about 6 hours. After this time the reaction was complete and the product essentially identical to that made in Example 1. For small scale preparations of the compounds described below, this simplified method was generally used.

SE2-SE2O Further alkenylsuccinic esters of pentaerythritol ethoxylates

The title compounds were made by the method described in Synthesis Example SE1, but varying the proportions of reagents and substituting the corresponding alkenyl succinic anhydride for the dodecenyl succinic anhydride used in SE1 to make the title compounds listed below. As triglycerol is a liquid, it can be ethoxylated by direct reaction with ethylene oxide under alkali catalysis without needing to use the two stage technique used for pentaerythritol.

The products were all obtained in quantitative yield as liquids or waxy solids. The identity of the products was confirmed by C¹³ and H¹ NMR. The products of these Examples were (compounds where the value of m is non-integral are indicated as the x(non-integral)-(alkenyl succinic acid)ester):

SE2—3.9-(dodecenyisuccinic)ester of pentaerythritol 48-ethoxylate

SE3—di-(dodecenylsuccinic)ester of pentaerythritol 158-ethoxylate

SE4—3.9-(dodecenylsuccinic)ester of pentaerythritol 15B-ethoxylate

SE5—di-(octadecenylsuccinic)ester of pentaerythritol 48-ethoxylate

SE6—3.9-(octadecenylsuccinic)ester of pentaerythritol 48-ethoxylate

SE7—di-(octadecenyisuccinic)ester of pentaerythritol 158-ethoxylate

SE8—3.9-(octadecenylsuccinic)ester of pentaerythritol 158-ethoxylate

SE9 tri-(tetradecenylsuccinic)ester of triglycerol 89-ethoxylate

SE10 4.9-(tetradecenyisuccinic)ester of triglycerol 89-ethoxylate

SE11 tri-(tetradecenylsuccinic)ester of triglycerol 135-ebioxylate

SE12 4.9-(tetradecenylsuccinic)ester of tnglycerol 135-ethoxylate

SE13 tri-(tetradecenylsuccinic)ester of triglycerol 169-ethoxylate

SE14 4.9-(tetradecenylsuccinic)ester of triglycerol 169-ethoxylate

SE15 tri-(octadecenylsuccinic)ester of triglycerol 89-ethoxylate

SE16 4.9-(octadecenylsuccinic)ester of triglycerol 89-ethoxylate

SE17 tri-(octadecenylsuccinic)ester of triglycerol 135-ethoxylate

SE18 4.9-(octadecenylsuccinic)ester of triglycerol 135-el,hoxylate

SE19 tri-(octadecenylsuccinic)ester of triglycerol 169-ethoxylate

SE20 4.9-(octadecenylsuccinic)ester of triglycerol 169-elhoxylate

SE21 2.9-(octadecenylsuccinic)ester of glycerol 120-ethoxylate

The title compound was made by the general method of SE1 but substituting glycerol 120-ethoxylate for the pentaerithritol-48 ethoxylate and octadecenyl succinic anhydride for the dodecenyl succinic anhydride used in SE1 and changing the molar proportions of the reagents to make the title compound. The intermediate glycerol 120-ethoxylate was prepared by direct reaction of glycerol and ethylene oxide under alkali catalysis; at about 120° C. The product was obtained as a waxy solid (melting at about 60° C.) in quantitative yield. The identity of the product was confirmed by C¹³ and H¹ NMR.

SE22 to SE29-(octadecenylsuccinc esters of sorbitol ethoxylates

The title compounds were made by the general method of SE1 but substituting sorbitol 80-ethoxylate and sorbitol 180-ethoxylate for the pentaerythritol ethoxylate and octadecenyl succinic anhydride for the dodecenyl succinic anhydride used in SE1 and changing the molar proportions of the reagents to make the title compounds. The products were obtained as waxy solids in quantitative yield and their respective identities confirmed by C¹³ and H¹ NMR. The products of these Examples were:

SE22 tri-(octadecenylsuccinic)ester of sorbitol 90-ethoxylate

SE23 hexa-(octadecenylsuccinic)ester of sorbitol 90-ethoxylate

SE24 tri-(octadecenyisuccinic)ester of sorbitol 180-ethoxylate

SE25 hexa-(octadecenylsuccinic)ester of sorbitol 180-ethoxylate

SE26 tri-(octadecenyisuccinic)ester of sorbitol 130-etho)ylate

SE27 hexa-(octadecenyisuccinic)ester of sorbitol 130-ethoxylate

SE28 tri-(octadecenylsuccinic)ester of sorbitol 220-etho)ylate

SE29 hexa-(octadecenylsuccinic)ester of sorbitol 220-ethoxylate

In practice, in these Examples, water was used to dissolve the sorbitol prior to ethoxylation so that the ethoxylates were mixtures of the respective sorbitol ethoxylates and polyoxyethylene glycol (PEG). The levels of ethoxylation indicated represent the total amount of ethylene oxide consumed in the ethoxylation based on the sorbitol used. Accordingly the ester products are in, effect, a mixture of the sorbitol ethoxylate succinic tri- or hexa-ester and the corresponding PEG succinic diester.

SE30 to SE35—Various esters of ethylene diamine PO/EO Block polyalkoxylate

The title compounds were made by the general method of SE21 but substituting ethylene diamine PO/EO (94/90) block polyalkoxylate for the glycerol ethoxylate and the appropriate alkenyl succinic anhydride for the dodecenyl succinic anhydride used in SE21 and changing the molar proportions of the reagents to make the title compounds. The intermediate block polyalkoxylate was made by reacting ethylene diamine tetrapropoxylate (1 PO unit condensed onto each amino active hydrogen) with propylene oxide using KOH as catalyst at about 125° C. to make the 94 mole PO condensate and subsequently feeding ethylene oxide to the reaction mix for at ime and in an amount to make the block polyalkoxylate. The title products were obtained as waxy solids in quantitative yield (based on the alkoxylates) and their respective identities confirmed by C¹³ and H¹ NMR. The products of these Examples were:

SE30 3.9-(octadecenylsuccinic)ester of ethylene diamine PO/EO (94/90) block polyalkoxylate

SE31 3.9-(octadecenylsuccinic)ester of ethylene diamine POIEO (94/140) block polyalkoxylate

SE32 3.9-(octadecenyisuccinic)ester of ethylene diamine PO/EO (94/180) block polyalkoxylate

SE33 3.9-(hexadecenyisuccinic)ester of ethylene diamine POIEO (94/90) block polyalkoxylate

SE34 3.9-(hexadecenylsuccinic)ester of ethylene diami'le POIEO (94/140) block polyalkoxylate

SE35 3.9-(hexadecenylsuccinic)ester of ethylene diamine POIEO (94/180) block Polyalkoxylate

SE36-SE39 Various alkenyl succinic esters of glycerol ethoxylates

The title compounds were made by the general method described in Example SE1 above but using appropriate glycerol ethoxylates for the pentaerithritol-48 ethoxylate used in SE1 and the appropriate alkenyl succinic anhydrides and adjusting the molar proportions of the reagents to make the title compounds. The intermediate glycerol ethoxylates were prepared by direct reaction of glycerol and ethylene oxide under alkali catalysis at about 120° C. The products were obtained as waxy solids (melting at about 60° C.) in quantitative yield and their identity was confirmed by C¹³ and H¹ NMR.

The title compounds of these Examples are:

SE36 2.9-(dodecenylsuccinic)ester of glycerol 44-ethoxylate

SE37 2.9-(dodecenyisuccinic)ester of glycerol 61-ethoxylate

SE38 2.9-(tetradecenyisuccinic)ester of glycerol 61-ethoxytate

SE39 2.9-(octadecenylsuccinic)ester of glycerol 61-ethoxylate

SE40 and SE41 alkenylsuccinic diesters of polyethylene glycol

The title compounds were made by the general method of Example SE1 but using polyethylene glycol (PEG) having a stated average molecular weight instead of the pentaerithritol-48 ethoxylate used in SE1 and using octadecenyl succinic anhydride instead of the dodecenyl succinic anhydride used in SE1 and adjusting the molar proportions to obtain the desired title compound. The products were all obtained in quantitative yield as liquids or waxy solids. The identity of the products was confirmed by C¹³ and H¹ NMR.

SE40 dioctadecenylsuccinic ester of PEG 4500

SE41 dioctadecenylsuccinic ester of PEG 5000

### APPLICATION EXAMPLES AE1 to AE9

A base shampoo was made up having the following composition:

| Material | Parts by weight |
|---|---|
| Tween 20 | 10.0 |
| Miranol C2M | 13.2 |
| Steol CS-330 | 17.2 |
| deionized water | 57.0 |
| Dowcil 200 | 0.1 |
| Total | 97.5 |

The pH of this base was adjusted to between 7 and 7.5. To this base was added 2.5 parts by weight of thickener and then the pH of that mixture was adjusted to between 6 and 7 with 50% aqueous citric acid. The test samples were stored for 24 hours (at least) in a 25° C. water bath. Viscosity measurements were then taken using a Brookfield Model LVDV1 viscometer. The thickeners for AE1 to AE9 were selected from those of SE1 to SE8 and SE21. A comparative Example AEC1 was included using 2.5 parts by PEG distearate (mean of 3 tests). The structures of the thickeners and the results of viscosity testing are set out in Table A1 below.

TABLE A1

| | Thickener | | | | | Viscosity |
|---|---|---|---|---|---|---|
| AE No | SE No | ASA | ester no | core | EO no | (mPa · s) |
| AEC1 | — | — | — | — | — | 175 |
| AE1 | SE1 | C12 | 2 | PE | 48 | 13.7 |
| AE2 | SE2 | C12 | 3.9 | PE | 48 | 199.2 |
| AE3 | SE3 | C12 | 2 | PE | 158 | 158 |
| AE4 | SE4 | C12 | 3.9 | PE | 158 | 21 |
| AE5 | SE5 | C18 | 2 | PE | 48 | 13.5 |
| AE6 | SE6 | C18 | 3.9 | PE | 48 | 19.7 |
| AE7 | SE7 | C18 | 2 | PE | 158 | 116 |
| AE8 | SE8 | C18 | 3.9 | PE | 158 | 12700 |
| AE9 | SE21 | C18 | 2.9 | glycerol | 120 | 66.0 |

### APPLICATION EXAMPLES AE10 to AE21

A further set of alkenyl succinic esters of ethoxylated triglycerol thickeners was tested as described for Application Example AE1 above. The structures of the thickeners and the viscosity results are set out in Table A2 below.

TABLE A2

| | Thickener | | | | | Viscosity |
|---|---|---|---|---|---|---|
| AE No | SE No | ASA | ester No | core | EO No | (mPa · s) |
| AE10 | SE9 | C14 | 3 | tri-gly | 89 | 58.9 |
| AE11 | SE10 | C14 | 4.9 | tri-gly | 89 | 89.2 |
| AE12 | SE11 | C14 | 3 | tri-gly | 135 | 744 |
| AE13 | SE12 | C14 | 4.9 | tri-gly | 135 | 288.6 |
| AE14 | SE13 | C14 | 3 | tri-gly | 169 | 39.3 |
| AE15 | SE14 | C14 | 4.9 | tri-gly | 169 | 357 |
| AE16 | SE15 | C18 | 3 | tri-gly | 89 | 99.9 |
| AE17 | SE16 | C18 | 4.9 | tri-gly | 89 | 4180 |
| AE18 | SE17 | C18 | 3 | tn-gly | 135 | 52 |
| AE19 | SE18 | C18 | 4.9 | tri-gly | 135 | 2468 |
| AE20 | SE19 | C18 | 3 | tri-gly | 169 | 67.6 |
| AE21 | SE20 | C18 | 4.9 | tri-gly | 169 | 3402 |

### APPLICATION EXAMPLES AE22 to AE29

A set of alkenyl succinic esters of sorbitol ethoxylates was tested as thickeners as described for Application Examples AE1 to AE9 above. The structures of the thickeners and viscosity results are set out in Table A3 below.

TABLE A3

| | Thickener | | | | Viscosity |
|---|---|---|---|---|---|
| AE No | SE No | ASA | ester No | EO No | (mPa · s) |
| AE22 | SE22 | 18 | 3 | 90 | 23 |
| AE23 | SE23 | 18 | 6 | 90 | 94.5 |
| AE24 | SE24 | 18 | 3 | 180 | 29.7 |
| AE25 | SE25 | 18 | 6 | 180 | 257 |
| AE26 | SE26 | 18 | 3 | 130 | 34 |
| AE27 | SE27 | 18 | 6 | 130 | 554 |
| AE28 | SE28 | 18 | 3 | 220 | 40 |
| AE29 | SE29 | 18 | 6 | 220 | 1198 |

### APPLICATION EXAMPLES AE30 to AE35

A set of alkenyl succinic esters of ethylene diamine alkoxylates was tested as thickeners as described for Application Examples AE1 to AE9 above. The structures of the thickeners and the viscosity results are set out in Table A4 below.

TABLE A4

| | Thickener | | | | | | Viscosity |
|---|---|---|---|---|---|---|---|
| AE No | SE No | ASA | ester No | core | PO No | EO No | (mPa · s) |
| AE30 | SE30 | C18 | 3.9 | eth | 94 | 90 | 19.6 |
| | | | | diam | | | |
| AE31 | SE31 | C18 | 3.9 | eth | 94 | 140 | 105.3 |
| | | | | diam | | | |
| AE32 | SE32 | C18 | 3.9 | eth | 94 | 180 | 86.6 |
| | | | | diam | | | |
| AE33 | SE33 | C16 | 3.9 | eth | 94 | 90 | 379 |
| | | | | diam | | | |
| AE30a | SE30 | C18 | 3.9 | eth | 94 | 90 | 1115 |
| | | | | diam | | | |
| AE34 | SE34 | C16 | 3.9 | eth | 94 | 140 | 1265 |
| | | | | diam | | | |
| AE31a | SE31 | C18 | 3.9 | eth | 94 | 140 | 3960 |
| | | | | diam | | | |
| AE35 | SE35 | C16 | 3.9 | eth | 94 | 180 | 1503 |
| | | | | diam | | | |
| AE32a | SE32 | C18 | 3.9 | eth | 94 | 180 | 3230 |
| | | | | diam | | | |

### APPLICATION EXAMPLES AE36 to AE40

The thickening properties of compounds of the invention in aqueous formulation were compared with conventional amine oxide surfactant thickeners. The formulations tested included varying amounts of sodium chloride to asses the effect of low to moderate electrolyte concentrations on the thickening properties of the materials. The basic formulation used was as follows:

| Material | | Parts by weight |
|---|---|---|
| SLES | | 15 |
| thickener | | 4.5 |
| salt | variable | 0, 1 or 2 |
| water | | 80.5 |

A similar comparison formulation was made up as AEC2 but containing 15 parts of CDMO and 66 parts of water. The structures of the thickeners amounts, of salt used and the results of viscosity testing are set out in Table A6 below.

TABLE A6

| | | | | | | Viscosity (mPa · s) | | |
|---|---|---|---|---|---|---|---|---|
| | Thickener | | | | | salt amount (g) | | |
| AE No | SE No | ASA C No | ester No | Core | EO No | 0 | 1 | 2 |
| AEC2 | — | — | — | — | — | 12 | 150 | 320 |
| AE36 | SE18 | C18 | 4.9 | tri-gly | 135 | 10200 | 3400 | 216000 |
| AE37 | SE20 | C18 | 4.9 | tri-gly | 169 | 252000 | — | — |
| AE38 | SE14 | C14 | 4.9 | tri-gly | 169 | 3200 | 6000 | 6400 |
| AE39 | SE2 | C12 | 3.9 | PE | 48 | 40 | 20 | 40 |
| AE40 | SE4 | C12 | 3.9 | PE | 158 | 840 | * | * |

*—2 phases

### APPLICATION EXAMPLES AE41 to AE47

Various compounds of the invention were tested for their ability to disperse titanium dioxide pigment in an aqueous formulation. The pigment formulation had the following composition:

| Material | Parts by weight |
|---|---|
| TR92 | 65 |
| water | 35 |
| dispersant | 1.5 |

The viscosity of the pigment dispersions was measured using a Brookfield LVT viscometer at 6 rpm (0.1 Hz). The results are set out in Table A7 below.

TABLE A7

| | Dispersant | | | | | Viscosity |
|---|---|---|---|---|---|---|
| AE No | SE No | ASA C No | ester No | Core | EO No | (mPa · s) |
| AE41 | SE36 | C12 | 2.9 | glycerol | 44 | 100 |
| AE42 | SE37 | C12 | 2.9 | glycerol | 61 | 49 |
| AE43 | SE38 | C14 | 2.9 | glycerol | 61 | 12 |
| AE44 | SE39 | C18 | 2.9 | glycerol | 61 | 12 |

### APPLICATION EXAMPLES TO AE45 to AE47

Various diesters of PEG were tested as thickeners in shampoo formulations. The formulations tested were based on a mild shampoo base and comparison runs were made using a shampoo made by thickening the shampoo base with 0.5% by weight of PEG 6000 distearate (AEC3) and with a commercially available proprietary shampoo using a similar shampoo base thickened with PEG distearate (AEC4). The viscosity of the thickened shampoos was measured at various shear rates in the range 10 to 100 s⁻¹. The materials used are set out in Table A8a, the measured viscosities of the formulations in Table A8b and the viscosities as a percentage of the measured viscosity at a shear rate of 10 s⁻¹ are set out in Table 8c below. These data indicate that the compounds of and used in this invention give higher viscosities at comparable levels of addition and exhibit desirably greater shear thinning that the conventional thickener (PEG 6000 distearate).

Table A8a

| | Thickener | | | | Amount (wt % on |
|---|---|---|---|---|---|
| AE No | SE No | ASA C No | ester No | Core | shampoo base) |
| AEC3 | PEG distearate | | | | 0.5 |
| AEC4 | PEG distearate | | | | (not known) |
| AE45 | SE40 | C18 | 2 | PEG 4500 | 0.5 |
| AE46 | SE41 | C18 | 2 | PEG 5000 | 1 |
| AE47 | SE41 | C18 | 2 | PEG 5000 | 2.5 |

TABLE A8b

| | Viscosity (m.Pa · s) | | | | | | | | | |
|---|---|---|---|---|---|---|---|---|---|---|
| | Shear rate (s⁻¹) | | | | | | | | | |
| AE No | 10 | 20 | 30 | 40 | 50 | 60 | 70 | 80 | 90 | 100 |
| AEC3 | 2310 | 2286 | 2236 | 2162 | 2117 | 2047 | 2007 | 1943 | 1864 | 1817 |
| AEC4 | 877.9 | 868.1 | 850 | 8749.1 | 845.3 | 820.7 | 811 | 802.9 | 800.8 | 791.1 |
| AE48 | 3558 | 3526 | 3417 | 3251 | 3077 | 2908 | 2767 | 2602 | 2505 | 2398 |
| AE49 | 3960 | 3345 | 3234 | 3133 | 2972 | 2789 | 2684 | 2537 | 2432 | 2324 |
| AE50 | 4242 | 3982 | 3867 | 3738 | 3569 | 3445 | 3310 | 3188 | 3100 | 3013 |

| | Table A8c | | | | | | | | | |
|---|---|---|---|---|---|---|---|---|---|---|
| | Viscosity as a percentage low shear viscosity | | | | | | | | | |
| | Shear rate (s⁻¹) | | | | | | | | | |
| AE No | 10 | 20 | 30 | 40 | 50 | 60 | 70 | 80 | 90 | 100 |
| AEC3 | 100 | 99 | 96.8 | 93.6 | 91.6 | 88.6 | 86.9 | 84.1 | 80.7 | 78.7 |
| AEC4 | 100 | 98.9 | 96.8 | 96.7 | 96.3 | 93.5 | 92.4 | 91.5 | 91.2 | 90.1 |
| AE48 | 100 | 98.3 | 95.2 | 90.6 | 85.8 | 81 | 77.1 | 72.5 | 69.8 | 66.8 |
| AE49 | 100 | 84.5 | 81.7 | 79.1 | 75.1 | 70.4 | 67.8 | 64.1 | 61.4 | 58.7 |
| AE50 | 100 | 93.9 | 91.2 | 88.1 | 84.1 | 81.2 | 78 | 75.2 | 73.1 | 71 |

### APPLICATION EXAMPLE AE48

Shampoo base/showergel type compositions were made up using various thickeners in the following formulation:

| Material | Parts by weight |
|---|---|
| Arlatone 1489 | 10 |
| Tensiomild HM | 20 |
| Tengobetain L7 | 10 |
| Germaben II | 2 |
| Thickener | 2 |
| Water | to 100 |

The thickener of SE7 was compared with G 1821 a commercially available PEG 6000 distearate and a commercially available proprietary tetrastearate thickener. The results are given in Table A9 below.

TABLE A9

| | Dispersant | | | | | Viscosity |
|---|---|---|---|---|---|---|
| AE No | SE No | ASA C No | ester No | Core | EO No | (mPa · s) |
| AEC5 | G 1821 (PEG 6000 distearate) | | | | | 200 |
| AEC6 | Crothix (pentaerythritol ethoxylate tetrastearate) | | | | | 35000 |
| AE48 | SE7 | 18 | 3.9 | PE | 158 | 65000 |

What is claimed is:

1. Compounds of the formula (I):
R².[(AO)ₙ.R³]ₘ (I)
where:
R² is the residue of a group having at least m active hydrogen atoms derived from hydroxyl and/or amino and/or amido groups;
AO is an alkylene oxide residue, which may vary along the chain;
each n is from 2 to 200;
m is from 2 to 10; and
each R³ is H, hydrocarbyl, long chain alk(en)yl succinic acyl group of the formula
OC.(HR)C.C(HR¹).COY
where:
one of R and R¹ in the succinic moiety is C₈ to C₂₂ alkenyl or alkyl and the other is hydrogen, and
Y is a group OM where M is hydrogen, metal, amonium, amine, especially alkylamine (including alkanolamine), onium, hydrocarbyl; or
Y is NR⁴R⁵ where R⁴ and R⁵ are each independently hydrogen, hydrocarbyl, particularly alkyl;
or a long chain acyl group —OC.R⁶, where R⁶ is a long chain hydrocarbyl group;
or a short chain acyl group —OC.R⁷, where R⁷ is a short chain hydrocarbyl group;
where at least two of the groups R³ are long chain acyl groups, and at least one of the long chain acyl groups is/are long chain alkenyl or alkyl succinic group(s);
provided that where R¹ is ethylene glycolyl or propylene glycolyl, m is 2, and both groups R² are alk(en)yl succinic groups, the total of the indices n is at least 120.

2. Compounds of the formula (Ia):
R².[(AO)ₙ.R³]ₘ (Ia)
where:
R² is the residue of a group having at least m active hydrogen atoms derived from hydroxyl and/or amino and/or amido groups;
AO represents ethylene oxide residues or a mixture of ethylene oxide residues and propylene oxide residues in which the molar proportion of ethylene oxide residues is at least 50% and desirably at least 70%;
each n is from 10 to 200 such that the total of the indices n is at least 120
m is from 2 to 10;
each R³ is H; hydrocarbyl;
a long chain alk(en)yl succinic acyl group of the formula:
—OC.(HR)C.C(HR¹).COY
where:
one of R and R¹ in the succinic moiety is C₈ to C₂₂ alkenyl or alkyl and the other is hydrogen, and
Y is a group OM where M is hydrogen, metal, amonium, amine, or
Y is NR⁴R⁵ where R⁴ and R⁵ are each independently hydrogen, a hydrocarbyl group;
a long chain acyl group —OC.R⁶, where R⁶ is a long chain hydrocarbyl group; or
a short chain acyl group —OC.R⁷, where R⁷ is a short chain hydrocarbyl group;
where at least two of the groups R³ are long chain acyl groups and at least one of the long chain acyl groups is/are long chain alkenyl or alkyl succinic group(s).

3. A compound as claimed in either claim 1 or claim 2 in which at least two of the groups R³ are long chain acyl groups and at least two of the long chain acyl groups is/are long chain alkenyl or alkyl succinic group(s).

4. A compound as claimed in claim 3 where at least three of the groups R³ are long chain acyl groups and at least three of the long chain acyl groups is/are long chain alkenyl or alkyl succinic group(s).

5. A compound as claimed in any one of claims 1 to 4 wherein, when R³ is hydrocarbyl it is a C₁ to C₂₂ alkyl or alkenyl group and when R³ is a long chain alk(en)yl succinic acyl group one of R⁴ and R⁵ is polyhydroxy substituted alkyl group.

6. A compound as claimed in claim 5 wherein one of R⁴ and R⁵ is an open chain tetratol, pentitol, hexitol or heptitol group or an anhydro derivative of such a group.

7. A compound as claimed in claim 5 wherein one of R⁴ and R⁵ is a residue of, or a residue derived from, glucose, fructose, maltose or palitose.

8. A compound as claimed in any one of claims 1 to 7 wherein the group R² is a residue of glycerol or a polyglycerol; a tri- or higher polymethylol alkane; a sugar, an etherifierd sugar; a partial alkyl ethers of a sugar; an ether oligo-/poly-mers of a sugar; a sugar ester; a polyhydroxy carboxylic acid; an amine; an amino-alcohol; a carboxylic acid amide; or an amido carboxylic acid.

9. A compound as claimed in claim 8 wherein the group R² is a residue of glycerol, diglycerol, triglycerol, trimethylol ethane, trimethylol propane, penterythritol, sorbitol, mannitol, sorbitan, methyl glucose, a dextrin, lauric, palmitic, oleic, stearic or behenic acid esters of sorbitan or a sorbitols, citric acid, tartaric acid, ethylene diamine 2-aminoethanol, di-ethanolamine, triethanolamine, urea, malonamide, succinamide, or succinamic acid.

10. A compound as claimed in any one of claims 1 to 9 wherein m is from 3 to 6.

11. A method of thickening an aqueous system which comprises including in the aqueous phase a thickening effective amount of a compound of the formula (II):
R¹².[(AO²)ₙ₂.R¹³]ₘ₂ (II)
where:
R¹² is the residue of an optionally substituted hydrocarbyl group having at least m active hydrogen atoms derived from hydroxyl and/or amino and/or amido groups;
AO² is an alkylene oxide residue, which may vary along the chain;
each n2 is from 10 to 200, such that the total of the indices n2 is at least 50;
m2 is from 2 to 10; and
each R¹³ is H, hydrocarbyl, a long chain alk(en)yl succinic acyl group of the formula:
—OC.(HR¹⁰)C.C(HR¹¹).COY²
where:
one of R¹⁰ and R¹¹ in the succinic acid moiety is C₈ to C₂₂ alkenyl or alkyl and the other is hydrogen, and
Y² is a group OM² where M² is hydrogen, metal, amonium, amine especially alkylamine, alkyl; or
Y² is NR¹⁴R¹⁵ where R¹⁵ and R¹⁵ are each independently hydrogen, a hydrocarbyl;
or a long chain acyl group —OC.R¹⁶, where R¹⁶ is a long chain hydrocarbyl group;
or a short chain acyl group —OC.R¹⁷, where R¹⁷ is a short chain hydrocarbyl group; where at least two of the groups R¹³ are long chain acyl groups, and at least one of the long chain acyl groups is a long chain alkenyl or alkyl succinic group.

12. A method as claimed in claim 9 wherein the compound of the formula (II) is a compound of the formula (I) or (Ia) as defined in any one of claims 1 to 7.

13. A method as claimed in claim 10 wherein the aqueous system is an oil-in-water emulsion, a water-in-oil emulsion, an aqueous solution or a dispersions of solids in an aqueous system.
